(19) United States
(12) Patent Application Publication
(10) Pub. No.: US 2022/0096191 A1
(43) Pub. Date: Mar. 31, 2022

(54) Caddy System for Equipment Sterilization

(71) Applicant: K1 Medical Technologies, LLC, Woodbridge, CT (US)

(72) Inventors: Walter J. Oko, Woodbridge, CT (US); Scott E. Cohen, Siesta Key, FL (US)

(73) Assignee: K1 Medical Technologies, Inc., Woodbridge, CT (US)

(21) Appl. No.: 17/429,095

(22) PCT Filed: Feb. 5, 2020
(86) PCT No.: PCT/US2020/016797

§ 371 Date: Aug. 6, 2021

Related U.S. Application Data
(60) Provisional application No. 62/801,818, filed on Feb. 6, 2019.

(51) Int. Cl. A61B 50/22; A61F 2/00; A61B 50/33

(52) U.S. Cl. CPC A61B 50/22; A61B 2050/3011; A61B 50/33; A61F 2/0095

(57) ABSTRACT

The present disclosure provides advantageous caddy system that is configured and adapted to promote modularity and withstand the harsh environment of central sterile processing processes. Caddy system may be removed and relocated on tray without additional fasteners or components. Caddy system may further provide identification features to correctly associate cataloged reusable medical devices to identified caddy systems and/or trays.

### CROSS-REFERENCE TO RELATED APPLICATION

The present application claims priority benefit to a U.S. provisional application entitled “Caddy System for Equipment Sterilization,” which was filed on Feb. 6, 2019, and assigned Ser. No. 62/801,818. The entire content of the foregoing provisional application is incorporated herein by reference.

### FIELD OF THE DISCLOSURE

The present disclosure generally relates to tray interfacing caddy system and, more generally, a sterilization tray and caddy system for detachably retaining reusable medical devices.

### BACKGROUND OF THE DISCLOSURE

Sterilization trays provide a container for retaining reusable medical devices during a patient's surgical procedure (e.g., during the perioperative process, which may include preoperative care, intraoperative care, and postoperative care) and/or during the central sterile processing process (e.g., autoclave-based steam sterilization). Included within the central sterile processing process are the steps of decontamination, cleaning, assemble and pack, sterilization, storage, distribution/transportation, and aseptic presentation. In hospitals and other health care facilities, a sterile processing department (e.g., Central Sterile Services Department) performs sterilization and other actions on medical devices, equipment, and consumables for subsequent use by medical professionals in the operating room of the hospital (or other health care facility) and also for other aseptic procedures.

Sterilization trays may include a cover, a base, and one or more attachment mechanisms for retaining reusable medical devices. After central sterile processing, sterilization trays may be brought into the operating room, or other venue, to provide a “tool box” for medical professionals to work out of. Despite being used by medical professionals as a “tool box”, sterilization trays provide no feedback, instruction, or ease of modularity to assist medical professionals

Based on the foregoing, a need exists for an effective modular “tool box” that offers flexibility and reliability in the positioning of reusable medical devices in connection with the perioperative process, the central sterile processing process, and/or subsequent access to/use of the sterilized reusable medical devices. Thus, an interest exists for modular sterilization trays and brackets, and related methods of use. These and other inefficiencies and opportunities for improvement are addressed and/or overcome by the assemblies, systems and methods of the present disclosure.

### SUMMARY OF THE DISCLOSURE

The present disclosure provides an advantageous assembly for detachably retaining devices. For example, for detachably retaining devices relative to a container (e.g., a caddy system, a tray). In particular, the present disclosure is directed to systems/methods for detachably retaining reusable medical devices and other instruments during the perioperative process and/or during the central sterile processing process. Even more particularly, exemplary assemblies are disclosed that include a tray with at least one caddy system positioned directly or indirectly thereto, for detachable retention of reusable medical devices and/or instruments to facilitate use/exposure during the perioperative process and/or the central sterile processing process.

As used throughout, the term “reusable medical device,” “medical device,” or any combination thereof, includes an instrument, apparatus, implement, machine, contrivance, implant, in vitro reagent, or other similar or related article, including a component part, accessory, lab equipment, reagent, or test kit, as will be known to a person skilled in the art. The above definition of a reusable medical device is intended to be broader than the definition provided by the United States Food and Drug Administration (e.g., https://www.fda.gov/MedicalDevices/DeviceRegulationandGuidance/Overview/ClassifyYourDevice/ucm051512.htm). Although the reusable medical device is referenced for use during surgery, additional instruments and/or devices that require central sterile processing, but are not used in connection with surgery, are expressly included in the present disclosure. Central sterile processing may include autoclave steam sterilization, which combines heat, humidity, and elevated pressure to sterilize a medical device. As used herein, a “user” generally refers to a human or a non-human and, in the case of a non-human, the term “user” generally refers to systems and devices that constitute and/or are controlled, in whole or in part, by software, artificial intelligence, robots and/or recognition software/machines

In exemplary embodiments, the present disclosure provides an advantageous caddy system that is configured and adapted to removably interface with (e.g., detachably interface with) a tray and further withstand the harsh environment of a central sterile processing process. The disclosed caddy system may further provide identification features to associate cataloged reusable medical devices to identified caddy systems and/or trays. The disclosed caddy system may be configured and adapted to removably interface with (e.g., detachably interface with) one or more modular post assemblies. Modular post assembly may include at least one post or at least one post and at least one bracket.

Any combination or permutation of features, functions and/or embodiments as disclosed herein is envisioned. Additional advantageous features, functions and applications of the disclosed systems, methods and assemblies of the present disclosure will be apparent from the description which follows, particularly when read in conjunction with the appended figures. All references listed in this disclosure are hereby incorporated by reference in their entireties.

### BRIEF DESCRIPTION OF DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features, steps and combinations of features/steps described below and illustrated in the figures can be arranged and organized differently to result in embodiments which are still within the scope of the present disclosure.

To assist those of ordinary skill in the art in making and using the disclosed assemblies, systems and methods, reference is made to the appended figures, wherein:

FIG. 1A schematically depicts a perspective view of an advantageous caddy system according to the present disclosure;

FIG. 1B schematically depicts a side view of an advantageous caddy system according to the present disclosure;

FIG. 2 schematically depicts a perspective view of an advantageous caddy system according to the present disclosure;

FIG. 3 schematically depicts a perspective view of an advantageous caddy system according to the present disclosure;

FIG. 4 schematically depicts a side view of an advantageous caddy system assembly with a plurality of medical devices, according to the present disclosure;

FIG. 5 schematically depicts a perspective view of an advantageous tray assembly with a plurality of caddy system assemblies, according to the present disclosure;

FIG. 6 schematically depicts a perspective view of a plurality of caddy system assemblies, according to the present disclosure;

FIG. 7 schematically depicts a side view of an advantageous tray assembly with a caddy system assembly, according to the present disclosure;

FIG. 8A schematically depicts a perspective view of an advantageous post according to the present disclosure;

FIG. 8B schematically depicts a side view of an advantageous post according to the present disclosure;

FIG. 9 schematically depicts a perspective view of an advantageous caddy system according to the present disclosure;

FIG. 10 schematically depicts a perspective view of an advantageous caddy system according to the present disclosure;

FIG. 11 schematically depicts a perspective view of an advantageous caddy system according to the present disclosure;

FIG. 12 schematically depicts a perspective view of an advantageous caddy system according to the present disclosure;

FIG. 13 schematically depicts a perspective view of an advantageous caddy system according to the present disclosure;

FIG. 14 schematically depicts a perspective view of an advantageous caddy system according to the present disclosure;

FIG. 15 schematically depicts a top view of a plurality of advantageous caddy systems according to the present disclosure;

FIG. 16 schematically depicts a perspective view of an advantageous tray assembly with a plurality of caddy system assemblies, according to the present disclosure;

FIG. 17A schematically depicts a top view of an advantageous tray assembly with a plurality of caddy system assemblies, according to the present disclosure;

FIG. 17B schematically depicts a perspective view of an advantageous tray assembly with a plurality of caddy system assemblies, according to the present disclosure;

FIG. 18A schematically depicts a perspective view of a caddy system assembly according to the present disclosure;

FIG. 18B schematically depicts a perspective view of a caddy system assembly according to the present disclosure;

FIG. 19A schematically depicts a top view of a caddy system assembly according to the present disclosure;

FIG. 19B schematically depicts a cross-sectional view of a caddy system assembly of FIG. 19A, according to the present disclosure;

FIG. 20A schematically depicts a side view of a caddy system assembly according to the present disclosure;

FIG. 20B schematically depicts a side view of a caddy system assembly according to the present disclosure;

FIG. 21 schematically depicts a side view of an advantageous tray assembly with two caddy system assemblies, according to the present disclosure;

FIG. 22 schematically depicts a side view of an advantageous tray assembly with two caddy system assemblies, according to the present disclosure;

FIG. 23 schematically depicts a top view of two caddy system assemblies according to the present disclosure;

FIG. 24 schematically depicts a top view of two caddy system assemblies according to the present disclosure;

FIG. 25A schematically depicts a perspective view of two caddy system assemblies according to the present disclosure; and

FIG. 25B schematically depicts a perspective view of two caddy system assemblies, in phantom, according to the present disclosure.

### DETAILED DESCRIPTION OF DISCLOSURE

The exemplary embodiments disclosed herein are illustrative of advantageous mounting/transporting assemblies (e.g., assemblies/devices for detachable retention of reusable medical devices during the perioperative process and/or during the central sterile processing process), and systems of the present disclosure and methods/techniques thereof. It should be understood, however, that the disclosed embodiments are merely illustrative of the present disclosure, which may be embodied in various forms. Therefore, details disclosed herein with reference to exemplary assemblies/fabrication methods and associated processes/techniques of assembly and use are not to be interpreted as limiting, but merely as the basis for teaching one skilled in the art how to make and use the advantageous assemblies/systems of the present disclosure.

The present disclosure provides an advantageous assembly for detachably retaining and/or containing and/or transporting reusable medical devices and other devices. For example, detachably retaining and/or containing and/or transporting reusable medical devices and other devices relative to a tray. In particular, the present disclosure is directed to systems/methods for detachably retaining reusable medical devices and other instruments during the perioperative process and/or during the central sterile processing process. Even more particularly, exemplary assemblies are disclosed that include a caddy system for detachable retention of reusable medical devices and/or instruments to facilitate use/exposure during the perioperative process and/or during the central sterile processing process. Even more particularly, at least one caddy system may removably interface with a tray, directly or indirectly thereto. The at least one caddy system may directly or indirectly detachably interface with at least one post positioned with respect to a tray.

The disclosed caddy system may include one or more features/elements to detachably interface with one or more reusable medical devices and other devices. The disclosed caddy system may include a plurality of features/elements to detachably interface with a plurality of reusable medical devices and other devices. The disclosed interfacing features may be fabricated with the disclosed caddy system. The disclosed interfacing features/elements may further include one or more retaining features/elements (e.g., boss features) to detachably retain the one or more reusable medical devices and other devices. The disclosed retaining features may be fabricated with the disclosed caddy system. The disclosed interfacing features may be configured and dimensioned to detachably retain one or more reusable medical devices and other devices perpendicular or angled with respect to a surface of the disclosed caddy system. The one or more reusable medical devices may be oriented with respect to the disclosed caddy system so as to promote efficient cleaning and sterilization of the reusable medical devices. The one or more retaining features may reduce the surface contact of the reusable medical device with respect to the disclosed caddy system, thereby promoting efficient cleaning and sterilization. The disclosed caddy system may detachably retain one or more medical devices that is/are oriented perpendicular with respect to a surface of the disclosed caddy system and one or more medical devices that is/are oriented angular with respect to a surface of the disclosed caddy system.

The disclosed interfacing features and/or the disclosed retaining features may be configured and adapted to releasably lock the one or more medical devices relative to the disclosed caddy system. The one or more medical devices may be initially positioned with respect to the one or more corresponding interfacing features and/or the retaining features. Then, the one or more medical devices may be repositioned to releasably lock relative to a surface of the disclosed caddy system. In a non-limiting example, the one or more medical devices may be positioned at least partially within the disclosed interfacing feature. Then, the one or more medical devices may be pivoted to releasably lock relative to a surface of the disclosed caddy system. The one or more medical devices may be pivoted to releasably lock upon at least partial engagement with one or more retaining features. The one or more medical devices may be initially positioned perpendicular with respect to a surface of the disclosed caddy system. Then, the one or more medical devices may be pivoted to a “locked position”. For example, the one or more medical devices may be oriented angular with respect to a surface of the disclosed caddy system. In the angled position, the one or more medical devices may be releasably locked with one or more retaining features. In one example, the one or more medical devices may be locked with respect to the disclosed caddy system to ensure the one or more medical devices remain positioned with respect to the disclosed caddy system during transportation.

The disclosed caddy system may further include one or more user engagement features/elements. The disclosed user engagement features/elements may promote ease of transportation of the disclosed caddy system. For example, the disclosed user engagement features may be fabricated with the disclosed caddy system.

The disclosed caddy system may be configured and dimensioned to include one “set” of reusable medical devices and other devices. For example, one set of reusable medical devices may include trial devices (e.g., femoral trial, reamer, tibial base template, tibial trials/sizers) for use in joint procedures. The disclosed caddy system may be customized to a user's preference (e.g., to include a plurality of medical device sizes). In an exemplary embodiment, a plurality of caddy systems may be positioned within one tray. The plurality of caddy systems may be configured and adapted to retain a plurality of variously-sized medical devices (e.g., femoral trial, reamer, tibial base template, tibial trials/sizers). The disclosed caddy system may include identification to notify the user the contents of the disclosed caddy system. For example, the disclosed identification may include unique characters (e.g., symbols, alphanumeric characters) and/or corresponding colors.

Referring now to the drawings, like parts are marked throughout the specification and drawings with the same reference numerals, respectively. Drawing figures are not necessarily to scale and in certain views, parts may have been exaggerated for purposes of clarity.

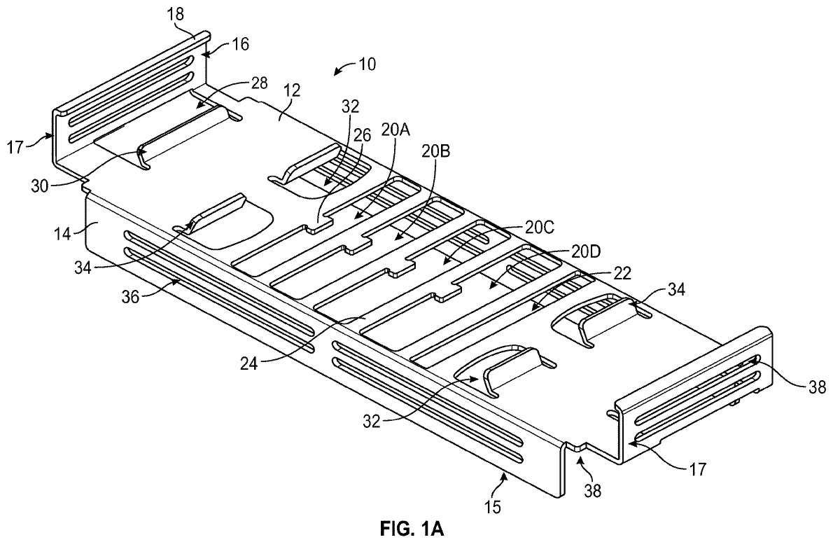

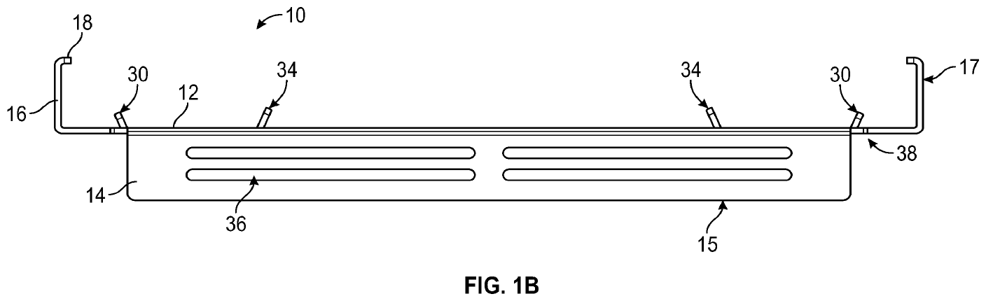

FIGS. 1A-1B schematically depict an advantageous caddy system 10 for detachably retaining and/or containing and/or transporting reusable medical devices and other devices (hereinafter “medical devices”). Caddy system 10 is defined, in part, by surface 12. Caddy system 10 may be further defined by sidewalls 14. Sidewalls 14 may extend angularly or perpendicularly from surface 12 so as to define a channel (e.g., U-shaped). U-shaped channel may be substantially (or partially) hollow so as to facilitate partial receipt of one or more medical devices. Surface 12 may further define at least one engagement feature for engaging at least one medical device. In an exemplary embodiment, surface 12 may further define a plurality of engagement features for engaging a plurality of medical devices.

Surface 12 may define at least one engagement feature 20, 22 having an opening extending through surface 12 into channel (e.g., U-shaped) defined below. Surface 12 may define a plurality of engagement features 20, 22 having corresponding openings extending through surface 12 into U-shaped channel defined below. Engagement feature 20, 22 may be configured and dimensioned to retain, at least in part, at least one medical device (e.g., tibial trials/sizers, tibial base template, reamer). The at least one medical device may be positioned at least partially within engagement feature 20, 22 and may be perpendicular or angled with respect to engagement features 20A-20D and/or surface 12. At least two engagement features 20, 22 may be separated by one or more ribs 24. Rib(s) 24 may partially increase the structural integrity of caddy system 12 (e.g., increased rigidity) and/or may directly/indirectly ensure that adjacent medical devices (e.g., tibial trials/sizers, tibial base template, reamer) do not touch. Rib(s) 24 may further define at least one boss feature 26. Boss feature 26 may be configured to partially traverse engagement feature 20, 22.

In an exemplary embodiment, surface 12 may define a plurality of tibial trial engagement features 20A-20D. Tibial trial engagement features 20A-20D may be configured and dimensioned to at least partially engage with one or more tibial trials. Tibial trials may be positioned at least partially within a corresponding engagement feature 20A-20D. In some instances, engagement features 20A-20D may be configured and dimensioned to at least partially engage with one or more variously-sized tibial trials. The corresponding engagement feature 20A-20D may be selected based, in part, on the dimensions (e.g., width, length, height) of the one or more tibial trials. For example, tibial trials having various thicknesses may interface with engagement features 20A-20D having an opening with similar dimensions so as to retain the variously-sized tibial trials (e.g., width of opening is similar to the thickness of the tibial trial). Tibial trials may be positioned perpendicular or angled with respect to engagement features 20A-20D and/or surface 12. Although depicted having four engagement features 20, more or less engagement features 20 are appreciated. Rib 24 may be positioned adjacent to engagement features 20. Rib(s) 24 may further define at least one boss feature 26. Boss feature 26 may be configured to partially traverse engagement feature 20. Boss feature 26 may partially interface with one or more tibial trials to ensure the tibial trial(s) maintain their predetermined position relative to engagement features 20A-20D and/or surface 12 (e.g., perpendicular, angled) and/or may directly/indirectly ensure adjacent medical devices do not contact each other. For example, boss feature 26 may position the one or more tibial trials to promote efficient cleaning and sterilization. Particularly, positioning the one or more tibial trials to have reduced contact with caddy system 10.

Caddy system 10 may further define at least one tibial base engagement feature 22. Tibial base engagement feature 22 may be configured and dimensioned to engage with one or more tibial base templates. Tibial base templates may be positioned at least partially within a corresponding engagement feature 22. In some instances, engagement feature 22 may be configured and dimensioned to at least partially engage with one or more variously-sized tibial base templates. The corresponding engagement feature 22 may be selected based, in part, on the dimensions (e.g., width, length, height) of the one or more tibial base templates. Tibial base templates may be positioned perpendicular or angled with respect to surface 12. Rib 24 may be positioned adjacent to engagement feature 22. Rib(s) 24 may further define at least one boss feature 26. Boss feature 26 may be configured to partially traverse engagement feature 22. Boss feature 26 may partially interface with one or more tibial base templates to ensure the tibial base template(s) maintain its/their position relative to engagement feature 22 and/or surface 12 (e.g., perpendicular, angled) and/or to directly/indirectly ensure adjacent medical devices do not contact each other. For example, boss feature 26 may position the at least one tibial base template to promote efficient cleaning and sterilization. Particularly, positioning the at least one tibial base template to have reduced contact with caddy system 10.

Caddy system 10 may further define one or more engagement features to at least partially engage with one or more femoral trials. For example, surface 12 may define, in part, opening 28 to engage with a first portion of the femoral trial and opening 32 to engage with a second portion of the femoral trial. Opening 28 and opening 32 may further include stabilization features 30, 34. Stabilization features 30, 34 may directly/indirectly interface with one or more surfaces of the femoral trial. Stabilization features 30, 34 may directly/indirectly interface with one or more contours of the femoral trial. Stabilization feature 30 may be positioned in close proximity to opening 28 and stabilization feature 34 may be positioned in close proximity to opening 34. Stabilization features 30, 34 may be perpendicular or angled with respect to opening 28, 32 and/or surface 12.

Caddy system 10 may further define one or more upright features 16 positioned with respect to surface 12. Upright feature 16 may be configured and dimensioned to interface with a sidewall of a tray. In a non-limiting example, upright feature 16 may be used as a handle for moving caddy system 10 and/or to directly/indirectly engage with one or more medical devices. However, additional uses are appreciated. Particularly, surface 17 of upright feature 16 may be positioned in close proximity to sidewalls of a tray. Upright feature 16 may further define feature 18 which extends from upright feature 16. Feature 18 may extend angularly or perpendicularly from upright feature 16. Upright feature 16 and feature 18, in combination, may define a handle. Feature 18 may define additional features for ease of handling and/or description of caddy system 10, including but not limited to, user engagement features (e.g., cut-outs, including square, rectangle, semi-circular, among other shapes) and unique characters (e.g., alphanumeric characters, symbols) and unique colors specific to one or more medical devices. Cut-outs may be positioned on opposing sides for engagement by a user. Although upright feature 16 and feature 18 are depicted as extending substantially the width of surface 12, upright feature 16 and/or feature 18 may be larger than or small than the width of surface 12. Upright feature 16 may, in part, define opening 28. Upright feature 16 may further define one or more openings 38 (e.g., slots). Slots 38 may facilitate pass-through of one or more fluids. For example, fluids associated with central sterile processing.

Sidewalls 14 of caddy system 10 may be configured and dimensioned to interface with one or more surfaces of a tray. For example, bottom edge 15 of sidewall 14 may be in direct/indirect contact with a bottom surface of a tray. Sidewalls 14 may further include one or more openings 36 (e.g., slots). Slots 36 may facilitate pass-through of one or more fluids. For example, fluids associated with central sterile processing.

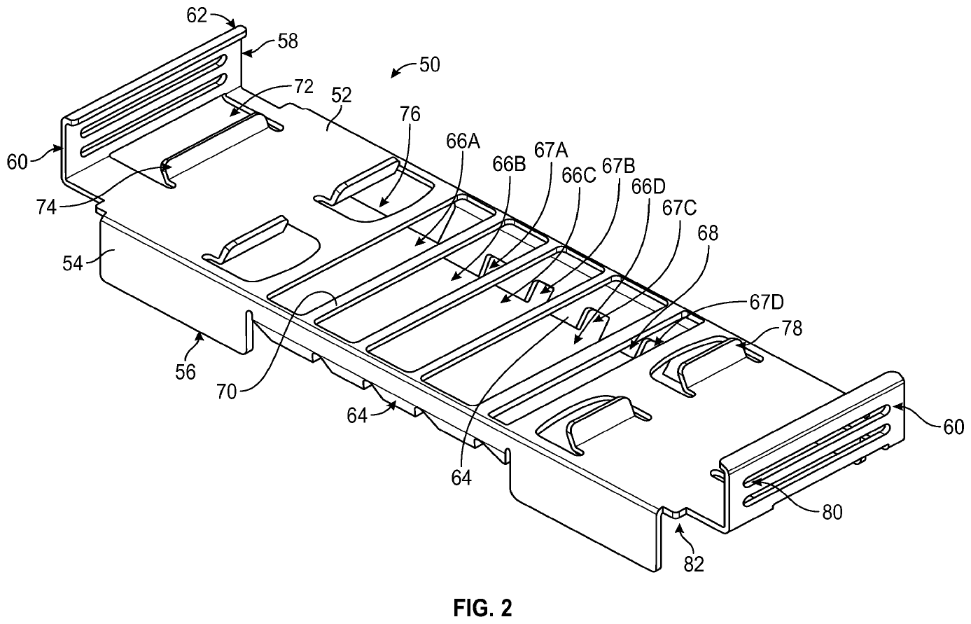

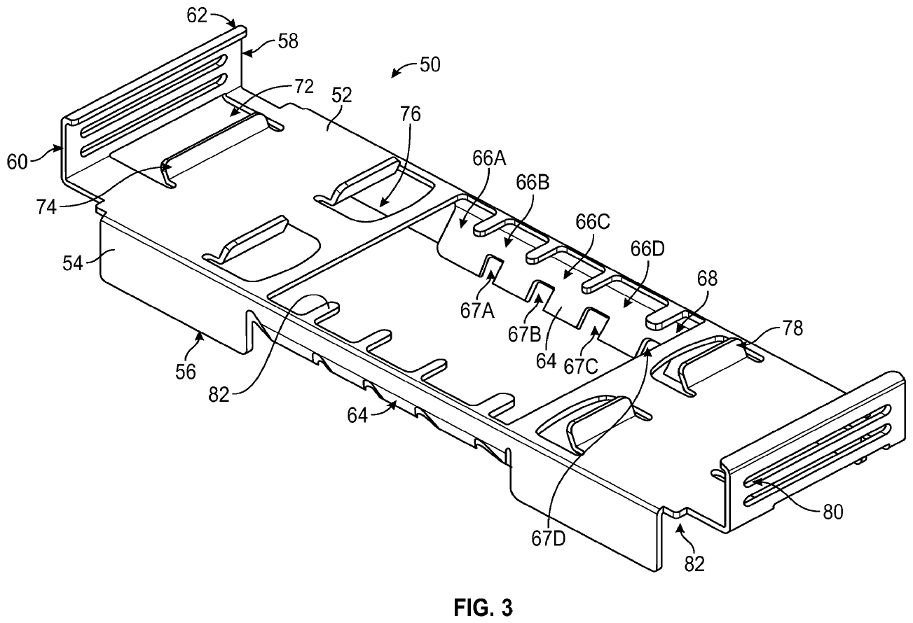

FIGS. 2 and 3 schematically depict an advantageous caddy system 50 for detachably retaining and/or containing and/or transporting reusable medical devices and other devices (hereinafter “medical devices”). Caddy system 50 is defined, in part, by surface 52. Caddy system 50 may be further defined by sidewalls 54. Sidewalls 54 extend angularly or perpendicularly from surface 52 so as to partially define a channel(e.g., U-shaped). U-shaped channel may be substantially (or partially) hollow so as to facilitate partial receipt of one or more medical devices. Surface 52 may further define at least one engagement feature for engaging at least one medical device. In an exemplary embodiment, surface 52 may further define a plurality of engagement features for engaging a plurality of medical devices.

Surface 52 may define at least one engagement feature 66, 68 having an opening extending through surface 52 into partial U-shaped channel defined below. Surface 52 may define a plurality of engagement features 66, 68 having corresponding openings extending through surface 52 into partial U-shaped channel defined below. Engagement feature 66, 68 may be configured and dimensioned to retain, at least in part, at least one medical device (e.g., tibial trials/sizers, tibial base template, reamer). The at least one medical device may be positioned at least partially within engagement feature 66, 68 and may be perpendicular or angled with respect to engagement features 66A-66D and/or surface 52. At least two engagement features 66, 68 may be separated by one or more ribs 70, 82. Rib(s) 70, 82 may partially increase the structural integrity of caddy system 52 (e.g., increased rigidity) and/or may directly/indirectly ensure that adjacent medical devices (e.g., tibial trials/sizers, tibial base template, reamer) do not touch. In some embodiments, rib(s) 70, 82 may further define at least one boss feature (not shown). Boss feature (not shown) may be configured to partially traverse engagement feature 66, 68.

In an exemplary embodiment, surface 52 may define a plurality of tibial trial engagement features 66A-66D. Tibial trial engagement features 66A-66D may be configured and dimensioned to at least partially engage with one or more tibial trials. Tibial trials may be positioned at least partially within a corresponding engagement feature 66A-66D. In some instances, engagement features 66A-66D may be configured and dimensioned to at least partially engage with one or more variously-sized tibial trials. The corresponding engagement feature 66A-66D may be selected based, in part, on the dimensions (e.g., width, length, height) of the one or more tibial trials. For example, tibial trials having various thickness may interface with engagement features 66A-66D having an opening with similar dimensions so as to retain the variously-sized tibial trials (e.g., width of opening is similar to the thickness of the tibial trial). Tibial trials may be positioned perpendicular or angled with respect to engagement features 66A-66D and/or surface 52. Although depicted having four engagement features 66, more or less engagement features 66 are appreciated. Rib 70, 82 may be positioned adjacent to engagement features 66. Rib(s) 70, 82 may further define at least one boss feature (not shown). Boss feature (not shown) may be configured to partially traverse engagement feature 66. Boss feature (not shown) may partially interface with one or more tibial trials to ensure the tibial trial(s) maintain their position relative to surface 52 (e.g., perpendicular, angled) and/or to directly/indirectly ensure adjacent medical devices do not contact each other. For example, boss feature (not shown) may position the one or more tibial trials to promote efficient cleaning and sterilization. Particularly, positioning the one or more tibial trials to have reduced contact with caddy system 50.

Caddy system 50 may further define at least one tibial base engagement feature 68. Tibial base engagement feature 68 may be configured and dimensioned to engage with one or more tibial base templates. Tibial base templates may be positioned at least partially within a corresponding engagement feature 68. In some instances, engagement feature 68 may be configured and dimensioned to at least partially engage with one or more variously-sized tibial base templates. The corresponding engagement feature 68 may be selected, in part, based on the dimensions (e.g., width, length, height) of the one or more tibial base templates. Tibial base templates may be positioned perpendicular or angled with respect to engagement feature 68 and/or surface 52. Rib 70, 82 may be positioned adjacent to engagement feature 68. As depicted in FIG. 2, rib 70 may be configured and dimensioned to substantially extend length-wise with engagement features 66A-66D, 68. As depicted in FIG. 3, rib 82 may be configured and dimensioned to partially extend length-wise with engagement features 66A-66D, 68. Rib(s) 70, 82 may further define at least one boss feature (not shown). Boss feature (not shown) may be configured to partially traverse engagement feature 68. Boss feature (not shown) may partially interface with one or more tibial base templates to ensure the tibial base template(s) maintain its/their position relative to surface 52 (e.g., perpendicular, angled) and/or to directly/indirectly ensure adjacent medical devices do not contact each other. For example, boss feature (not shown) may position the at least one tibial base template to promote efficient cleaning and sterilization. Particularly, positioning the at least one tibial base template to have reduced contact with caddy system 50.

Caddy system 50 may further define support feature 64. Support feature 64 may be positioned in close proximity to engagement features 66, 68. Support feature 64 may be positioned within U-shaped channel and below engagement features 66, 68 positioned with respect to surface 52. Support feature 64 may be perpendicular or angled with respect to engagement features 66A-66D, 68 and/or surface 52. Support feature 64 may further define openings 67A-67D. Although four openings 67A-67D are depicted, more or less openings are appreciated. Openings 67A-67D may be configured and dimensioned to directly/indirectly engage with one or more medical devices. For example, openings 67A-67D may correspond with engagement features 66A-66D and/or 68 and may be configured to engage with one or more tibial trials and/or tibial base template. The position of openings 67A-67D may depend, in part, on whether the one or more tibial trials/tibial base template are intended to be positioned perpendicular or angled with respect to engagement features 66A-66D, 68 and/or surface 52. For example, where one or more tibial trials/tibial base template are intended to be positioned perpendicular or angled with respect to engagement features 66A-66D, 68 and/or surface 52, openings 67A-67D may be aligned with or offset from engagement features 66A-66D, 68 positioned above.

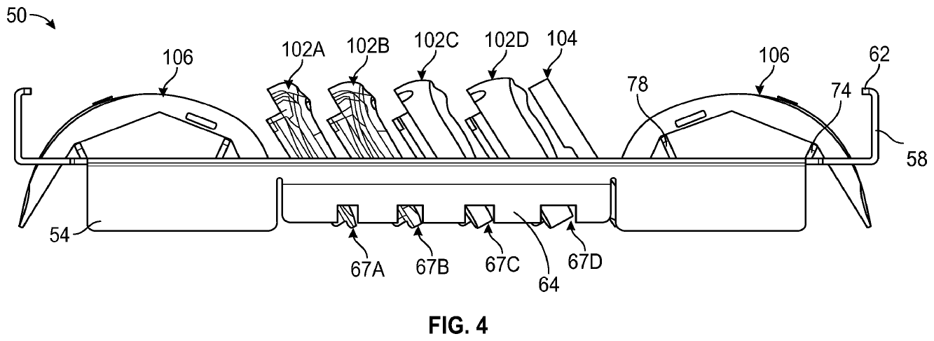

For instance, if tibial trials/tibial base template were intended to be perpendicular, as described above, openings 67A-67D may be aligned with engagement features 66A-66D, 68. Thus, engagement of tibial trials/tibial base template with their corresponding engagement features 66A-66D, 68 and openings 67A-67D would result in tibial trials/tibial base template being substantially perpendicular with respect to engagement features 66A-66D, 68 and/or surface 52. In another instance, if tibial trials/tibial base template were intended to be angled, as described above and depicted in FIG. 4, openings 67A-67D may be positioned offset from engagement features 66A-66D, 68. Thus, engagement of tibial trials 102A-102D and/or tibial base template 104 with their corresponding engagement features 66A-66D, 68 and openings 67A-67D would result in tibial trials/tibial base template being substantially angled with respect to engagement features 66A-66D, 68 and/or surface 52.

Caddy system 50 may further define one or more engagement features to at least partially engage with one or more femoral trials (See FIG. 4). For example, surface 52 may define, in part, opening 72 to engage with a first portion of the femoral trial and opening 76 to engage with a second portion of the femoral trial. Opening 72 and opening 76 may further include stabilization features 74, 78. Stabilization features 74, 78 may directly/indirectly interface with one or more surfaces of the femoral trial. Stabilization features 74, 78 may directly/indirectly interface with one or more contours of the femoral trial. Stabilization feature 74 may be positioned in close proximity to opening 72 and stabilization feature 78 may be positioned in close proximity to opening 76. Stabilization features 74, 78 may be perpendicular or angled with respect to opening 72, 76 and/or surface 52.

Caddy system 50 may further define one or more upright features 58 positioned with respect to surface 52. Upright feature 58 may be configured and dimensioned to interface with a sidewall of a tray. In a non-limiting example, upright feature 58 may be used as a handle for moving caddy system 50 and/or to directly/indirectly engage with one or more medical devices. However, additional uses are anticipated. Particularly, surface 60 of upright feature 58 may be positioned in close proximity to sidewalls of a tray. Upright feature 58 may further define feature 62 which extends from upright feature 58. Feature 62 may extend angularly or perpendicularly from upright feature 58. Upright feature 58 and feature 62, in combination, may define a handle. Feature 62 may define additional features for ease of handling and/or description of caddy system 50, including but not limited to, user engagement features (e.g., cut-outs, including square, rectangle, semi-circular, among other shapes) and unique characters (e.g., alphanumeric characters, symbols) and unique colors specific to one or more medical devices. Cut-outs may be positioned on opposing sides for engagement by a user. Although upright feature 58 and feature 62 are depicted as extending substantially the width of surface 52, upright feature 58 and/or feature 62 may be larger than or smaller than the width of surface 52. Upright feature 58 may, in part, define opening 72. Upright feature 58 may further define one or more openings 80 (e.g., slots). Slots 80 may facilitate pass-through of one or more fluids. For example, fluids associated with central sterile processing.

Sidewalls 54 of caddy system 50 may be configured and dimensioned to interface with one or more surfaces of a tray. For example, bottom edge 56 of sidewall 54 may be in direct/indirect contact with a bottom surface of a tray. Sidewalls 54 may further include one or more openings (not shown) (e.g., slots). Slots (not shown) may facilitate pass-through of one or more fluids. For example, fluids associated with central sterile processing.

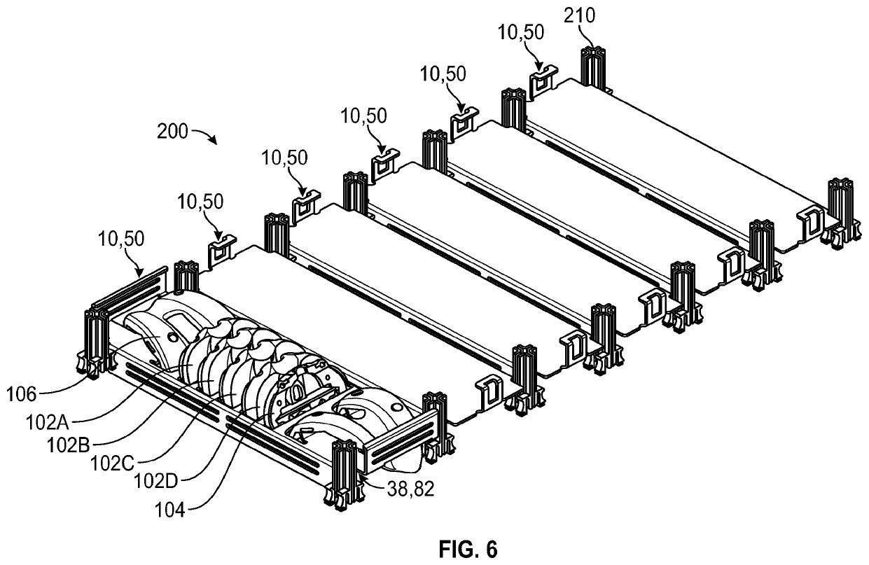

In another embodiment, tibial trials 102A-102D and/or tibial base template 104 may be initially positioned within corresponding engagement feature 20A-20D, 22. Tibial trials 102A-102D and/or tibial base template 104 may be initially positioned within corresponding engagement feature 20A-20D, 22 in a substantially perpendicular configuration. Then, tibial trials 102A-102D and/or tibial base template 104 may be pivoted relative to corresponding engagement feature 20A-20D and/or surface 12 to releasably lock tibial trials 102A-102D and/or tibial base template 104 relative to engagement feature 20A-20D, 22, as depicted in FIGS. 4-7. Tibial trials 102A-102D and/or tibial base template 104 may be releasably locked relative to surface 12. At least one boss feature 26 may, in part, releasably lock tibial trials 102A-102D and/or tibial base template 104 with respect to surface 12.

In another embodiment, tibial trials 102A-102D and/or tibial base template 104 may be initially positioned within corresponding engagement feature 66A-66D, 68. Tibial trials 102A-102D and/or tibial base template 104 may be initially positioned within corresponding engagement feature 66A-66D, 68 in a substantially perpendicular configuration. Then, tibial trials 102A-102D and/or tibial base template 104 may be pivoted relative to corresponding engagement feature 66A-66D and/or surface 52 to releasably lock tibial trials 102A-102D and/or tibial base template 104 relative to engagement feature 66A-66D, 68, as depicted in FIGS. 4-7. Tibial trials 102A-102D and/or tibial base template 104 may be releasably locked relative to surface 52. At least one boss feature (not shown) may, in part, releasably lock tibial trials 102A-102D and/or tibial base template 104 with respect to surface 52.

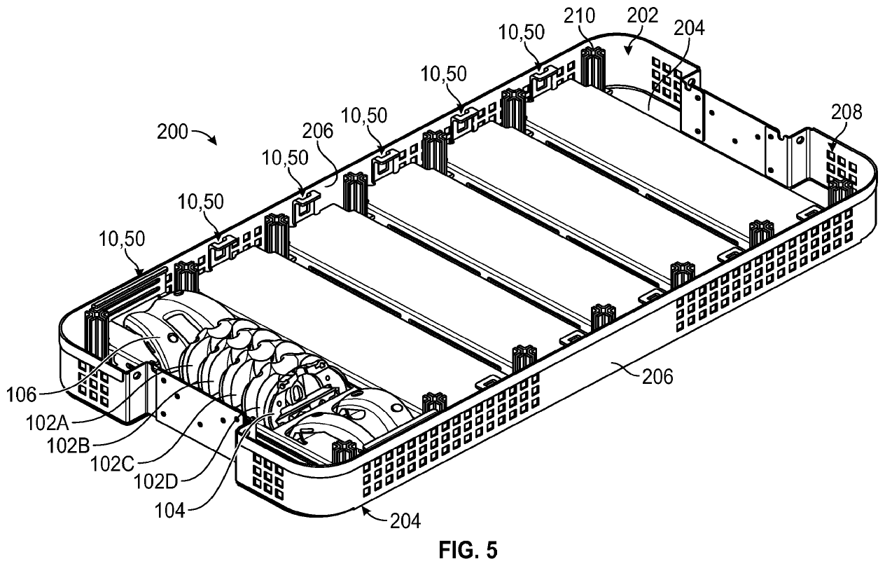

FIGS. 5-7 schematically depict assembly 200 including a plurality of caddy systems 10, 50 positioned adjacent to each other and with respect to tray 202. Although depicted as a plurality of caddy systems 10, 50, one or more cavity systems 10, 50 may be utilized without departing from the spirit/scope of this disclosure. Depending on the situation, tray 202 may be partially filled or completely filled with one or more caddy system(s) 10, 50.

Tray 202 includes bottom 204 and sidewalls 206 which partially define an interior. Interior is configured and dimensioned to receive one or more caddy systems 10, 50. Particularly, bottom edge 15, 56 of sidewall 14, 54 of caddy system 10, 50 may be at least partially in direct/indirect contact with bottom 204 of tray 202. More particularly, bottom edge 15, 56 may define features/elements that at least partially interface with bottom 204. For example, features/elements that enable fluid to pass-through (e.g., fluid from the central sterile process) and/or that interface caddy system 10, 50 with bottom 204. Tray 202 further includes a plurality of perforations 208 positioned with respect to bottom 204 and/or sidewalls 206. Perforations 208 may assist with securing one or more caddy system(s) 10, 50 to tray 202. However, caddy system 10, 50 may be positioned within the interior of tray 202 without being affixed either directly/indirectly by one or more perforations 208.

In an exemplary embodiment, caddy system 10, 50 may be positioned within the interior of tray 202. The quantity of caddy systems 10, 50 positioned within tray 202 may depend on the desired perioperative process and/or central sterile processing process. Thus, one caddy system 10, 50 may be positioned within tray 202 or a plurality of caddy systems 10, 50 may be positioned within tray 202. If two or more caddy systems 10, 50 are positioned within one tray 202, one or more caddy systems 10, 50 may be removed from tray 202 without disturbing one or more of the remaining caddy systems 10, 50. Caddy system 10, 50 may be in direct/indirect contact with one or more sidewalls 206 and bottom 204. Caddy system 10, 50 may be in direct/indirect contact with two sidewalls 206 and bottom 204. Caddy system 10, 50 may be in direct/indirect contact with two sidewalls 206, bottom 204 and at least one adjacent caddy system 10, 50.

Caddy system 10, 50 may be releasably contained within tray 202 by one or more posts 210 positioned with respect to one or more perforations 208. Particularly, one or more posts 210 may be in direct/indirect contact with one or more corners 38, 82 of caddy system 10, 50. In some embodiments, one post 210 may be in direct/indirect contact with at least two caddy systems 10, 50. In other embodiments, one post 210 may be in direct/indirect contact with up to four caddy systems 10, 50. Post 210 and/or caddy system 10, 50 (e.g., corner 38, 82) may include features/elements to facilitate engagement between post 210 and caddy system 10, 50.

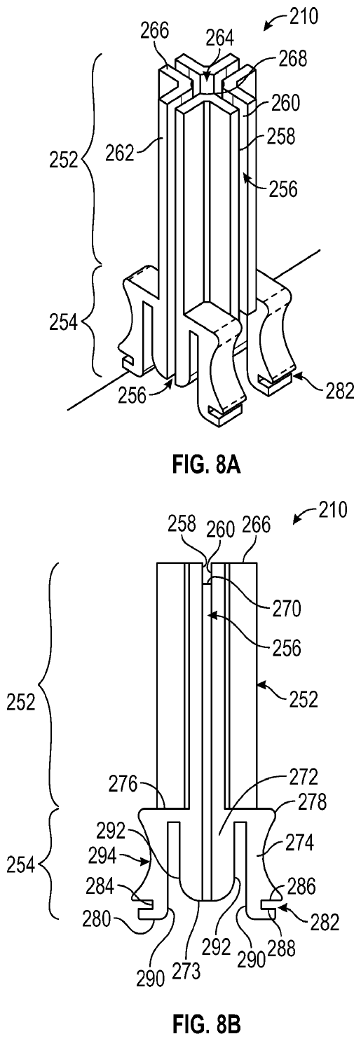

With reference to FIGS. 8A-8B, advantageous post 210 may include body 252 and tray engagement feature (e.g., clip) 254. Body 252 and clip 254 may be fabricated to form a unitary component (e.g., molding, casting, extruding) or may be assembled together from individual components. In one embodiment, where body 252 and clip 254 are a single fabricated component, some features of one component may extend through or blend into the other component. Therefore, it should be understood that the labels “body” and “clip” are merely explanatory and are not intended to rigidly define post 210.

Body 252 may be fabricated such that the ratio between the height and the cross-section favors the height. However, the ratio between the height and cross-section may be equal or may favor the cross-section, depending on the application, as will be evident from this disclosure. The cross-section of body 252 may be fabricated from a variety of shapes, including a quadrilateral (e.g., square, rectangle, diamond), a circle, a triangle, a balbis (“H-shape”), a cross (“plus sign”), among others, as will be apparent based on the intended purpose of this disclosure.

In an exemplary embodiment, body 252 has a height that is greater than the cross-sectional distance and a cross-section with a substantially cross shape (“plus sign”). Body 252 may further include at least one attachment feature. Attachment feature may include at least one slot 256 and optionally at least one hole 264. At least one slot 256 is defined by two side walls 258, 260. At least one hole 264 may be centrally located on top face 266 and extend some distance inward from top face 266 to clip portion 254. Slot 256 may extend some distance from top surface 266 towards clip 254. In some embodiments, slot 256 extends from top surface 266 through at least a portion of clip 254. The depth of slot 256 may be further defined by the distance between face 262 and slot bottom 268. Slot 256 may be recessed a distance below top face 266, such that slot top 270 is the top of slot 256. The cavity between slot top 270 and top face 266 is configured and dimensioned to accept a portion of partition, further described below. Slot 256, as defined by side walls 258, 260 and slot bottom 268, may be square, rectangle, semi-circular, among other shapes.

Body 252 may further include additional attachment features. For example, alternatively located holes, dimples, slots, cavities, fasteners, etc., as can be appreciated in view of this disclosure. It is a desire to illustrate that post 210 and at least a portion of caddy system 10, 50 may be releasably attached. As such, attachment features that provide attachment and detachment of at least a portion of caddy system 10, 50 from post 210 are appreciated. Attachment features may further include features for retaining one or more corners 38, 82 of caddy system 10, 50. Interfacing corners 38, 82 may be fixedly attached to post 210 and/or may be positioned in close proximity to post 210 so as to retain caddy system 10, 50 from at least partial movement without fixedly engaging caddy system 10, 50.

Clip 254 may include clip body 272 and two symmetrically adjacent arms 274, located on either side of clip body 272. Clip body 272 may be coplanar with face 262. In some embodiments, side walls 258, 260 and clip body 272 may be the same component, as depicted in FIGS. 8A-8B. In other embodiments, clip 254 may be a separate component. As mentioned above, slot 256 may extend at least partially through clip 254. In one embodiment, slot 256 may extend from top surface 266 through base 273 on clip body 272. Surface 276 connects arm 274 to clip body 272 by way of shoulder 278. Located above arm base 280 is cavity 282, which provides a tray engagement feature to directly or indirectly attach to at least one tray perforation 208. The disclosed interface features of cavity 282 may include back face 284, upper extension 286, and lower extension 288, which collectively form cavity 282. In relation to back face 284, the length of lower extension 288 may be shorter than the length of upper extension 286, as depicted. Lower extension 288 having a shorter length enables an easier installation and removal from tray perforations without sacrificing clip engagement. However, in another embodiment, upper extension 286 and lower extension 288 may have similarly dimensioned lengths.

In exemplary operation, at least a portion of cavity 282 captures a portion of a tray perforation. To ensure clip 254 does not shift after installation with tray 202, back face 284 is under spring load in contact with an inside wall of a perforation. Furthermore, upper extension 286 and lower extension 288 may be in close proximity to tray 202. Particularly, upper extension 286 and lower extension 288 may at least partially engage with at least one surface of tray 202. Even more particularly, upper extension 286 may at least partially engage with the interior surface of tray 202 and lower extension 288 may at least partially engage with the exterior surface of tray 202.

In one example, by simultaneously compressing arms 274, such that arms 274 at least partially flex, face 290 moves closer to outer face 292 on clip body 272 and the distance between back face 284 on either arm 274 is reduced. Reduction of the distance between back face 284 on either arm 274 enables cavity 282 to be engaged with the respective perforations. Once arms 274 are released into their relaxed position, back face 284 will be in contact with a desired engagement surface on the respective perforation. Particularly, since the resting distance between back faces 284 is greater than the distance between the engagement surfaces (e.g., inside wall), arms 274 remain in slight compression and therefore apply a spring load to the engagement surface of the respective perforation. In another example, by individually compressing arms 274, such that arms 274 at least partially flex, one cavity 282 may be engaged with the respective perforation. Upon engagement of one cavity 282, second cavity 282 may be engaged with the perforation. Aside from the method to engage clip 254 with a tray (e.g., individual arm compression or simultaneous arm compression), the engagement with the perforation is substantially similar. Therefore, arms 274 apply a spring load to the desired engagement surface on the respective perforation. Of note, attachment is not to be limited to a tray; rather, attachment may further be accomplished with the sidewalls and the cover (not shown). For purposes of the present disclosure, when one of a tray, a sidewall(s) and/or a cover are mentioned, the others not mentioned are included, unless otherwise stated.

In an exemplary embodiment, arms 274 may include a semi-circular feature 294 located on the outside surface of arm 274, opposite surface 290. Semi-circular feature 294 may provide a specific location for a user to place their fingers to assist in compressing arms 274 for insertion or removal from a tray.

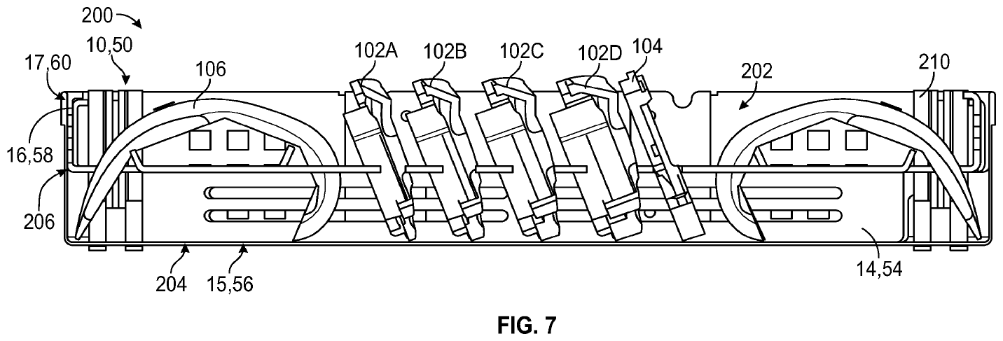

Caddy system 10, 50 may be configured and dimensioned so as to suspend medical devices a distance above bottom 204, as particularly depicted in FIG. 7. FIG. 7 depicts medical devices positioned on an angle, as thoroughly discussed above. Positioning medical devices on an angle enables taller medical devices to fit substantially with the interior of tray 202 such that a lid may be positioned on top of tray 202. Particularly, tibial trials 102A-102D and/or tibial base template 104 may be positioned on an angle. Positioning medical devices (e.g., tibial trials and tibial base template) perpendicularly or angularly, as described above, promotes better cleaning and sterilization, a shortcoming of current packaging configurations (e.g., medical devices positioned laying down).

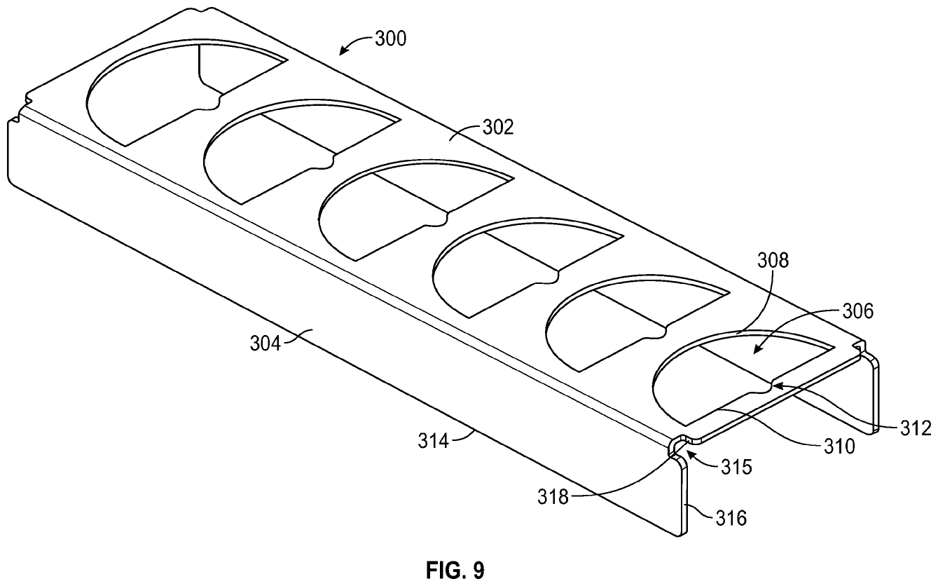

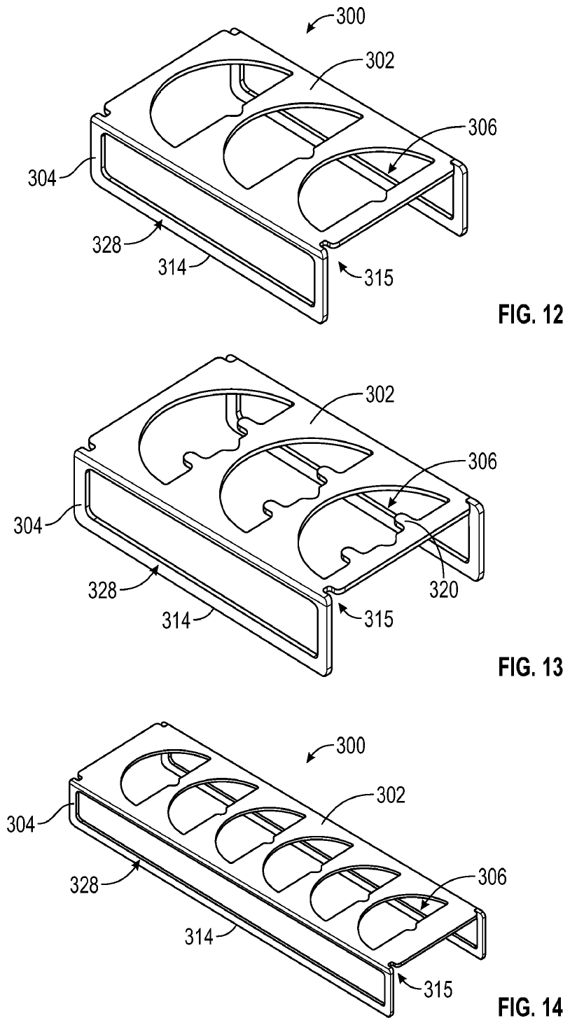

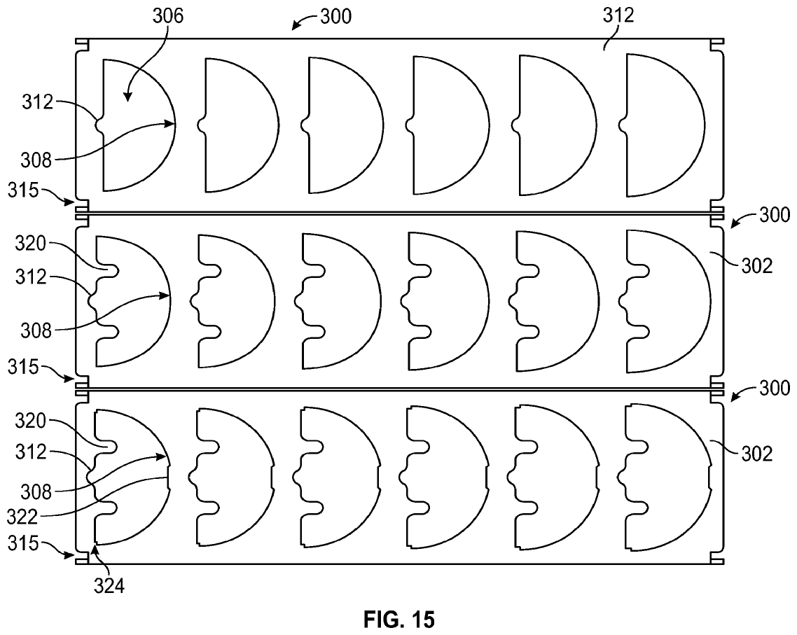

FIGS. 9-15 schematically depict an advantageous caddy system 300 for detachably retaining and/or containing and/or transporting reusable medical devices and other devices (hereinafter “medical devices”). Caddy system 300 is defined, in part, by surface 302. Caddy system 300 may be further defined by sidewalls 304. Sidewalls 304 extend angularly or perpendicularly from surface 302 so as to define a channel (e.g., U-shaped). U-shaped channel may be substantially (or partially) hollow so as to facilitate partial receipt of one or more medical devices. Surface 302 may further define at least one engagement feature for engaging at least one medical device. In an exemplary embodiment, surface 302 may further define a plurality of engagement features for engaging a plurality of medical devices. FIG. 15, specifically, depicts three advantageous engagement features side-by-side for ease of comparison.

Surface 302 may define at least one engagement feature 306 having an opening extending through surface 302 into channel (e.g., U-shaped) defined below. Surface 302 may define a plurality of engagement features 306 having corresponding openings extending through surface 302 into U-shaped channel, defined below. Engagement feature 306 may be configured and dimensioned to retain, at least in part, at least one medical device (e.g., tibial trials/sizers, tibial base template, reamer). The at least one medical device may be positioned at least partially within engagement feature 306 and may be perpendicular or angled with respect to engagement features 306 and/or surface 302. At least two engagement features 306 may be positioned a predetermined distance from each other. Thus, surface 302 may separate at least two engagement features 306, which may partially increase the structural integrity of caddy system 300 (e.g., increased rigidity) and/or may ensure that adjacent medical devices (e.g., tibial trials/sizers, tibial base template, reamer) do not touch. Engagement features 306 may further define at least one boss feature 320, 322. Boss feature 320, 322 may be configured to partially traverse engagement feature 306. Caddy system 300 may further define one or more openings 326, 328 (e.g., slots). Slots 326, 328 may facilitate pass-through of one or more fluids. For example, fluids associated with central sterile processing.

In an exemplary embodiment, surface 302 may define a plurality of reamer engagement features 306. Reamer engagement features 306 may be configured and dimensioned to at least partially engage with one or more reamers. Reamers may be positioned at least partially within a corresponding engagement feature 306. In some instances, engagement features 306 may be configured and dimensioned to at least partially engage with one or more variously-sized reamers. The corresponding engagement feature 306 may be selected based, in part, on the dimensions (e.g., width, length, height) of the one or more reamers. For example, reamers having various diameters and/or heights may interface with engagement features 306 having an opening (e.g., semispherical opening) with similar dimensions so as to retain the variously-sized reamers (e.g., width and diameter of opening are similar to the height and the diameter of the reamer, respectively). Caddy system 300 may engage with a plurality of variously-sized reamers. Reamers may be positioned perpendicular or angled with respect to engagement features 306 and/or surface 302. Although depicted having between three and six engagement features 306, more or less engagement features 306 are appreciated.

Engagement feature 306 may be a semispherical opening defining a curved edge 308 and a connecting edge 310. Semispherical opening of engagement feature 306 may be sized to at least partially engage with one or more reamers of corresponding size. Engagement feature 306 may further define recessed portion 312 for receipt of one or more reamer features and/or surfaces, as will be discussed in more detail below. Connecting edge 310 may define recessed portion 312, which may be positioned of equal distance between the outer edges of engagement feature 306.

Engagement feature 306 may further define one or more boss features 320, 322. Engagement feature 306 may define at least two boss features 320. Engagement feature 306 may define at least one boss feature 322. Boss feature 320, 322 may be configured to partially traverse engagement feature 306. Boss feature 320, 322 may partially interface with one or more reamers to ensure the reamer(s) maintain its/their position relative to engagement features 306 and/or surface 302 (e.g., perpendicular, angled) and/or to directly/indirectly ensure adjacent medical devices do not contact each other. For example, boss feature 320, 322 may position the one or more reamers to allow for enhanced cleaning. Particularly, positioning the one or more reamers to have reduced contact with caddy system 300.

Caddy system 300 may further include one or more tray engagement features 315. Tray engagement feature 315 may directly/indirectly engage with one or more features on tray 202. Tray engagement features 315 may interface with one or more posts 210, which may be mounted with respect to tray 202. Tray engagement feature 315 defines sidewall edge 316 and surface edge 318. Sidewall edge 316 may be configured and dimensioned to engage with slot 256 of post 210. Surface edge 318 may be configured to contact one or more surfaces of post 210. Caddy system 300 may interface with one or more posts 210. Caddy system 300 may interface with two or more posts 210. Caddy system 300 may interface with four posts 210, one in each corner of caddy system 300.

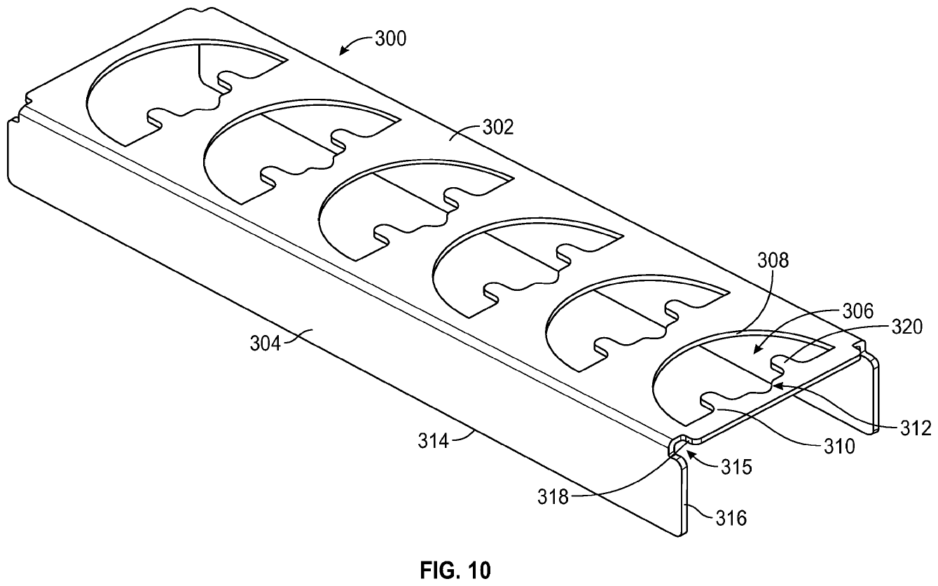

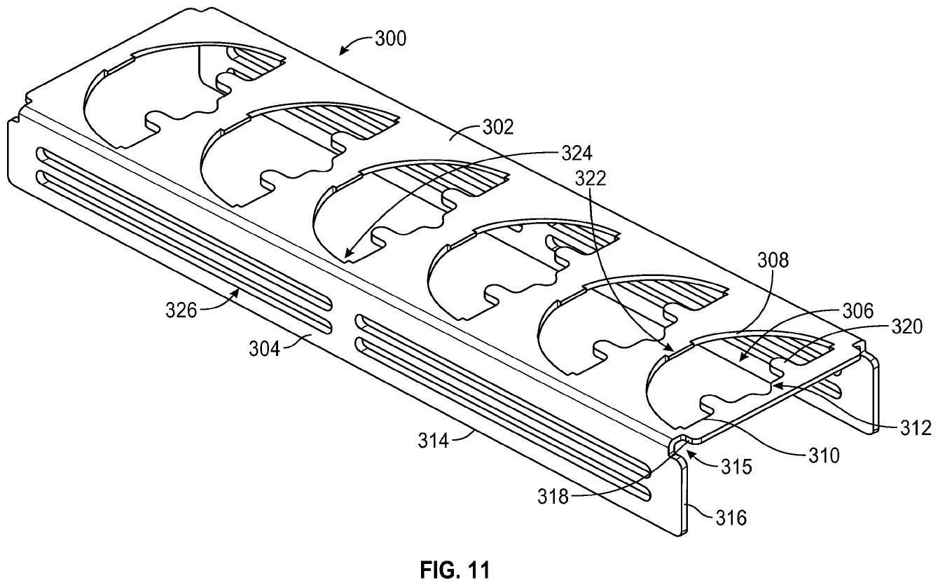

With specific reference to FIGS. 10 and 11, boss feature 320 may extend from connecting edge 310 and partially traverse engagement feature 306 a predetermined distance. Boss feature 320 may be configured to partially engage with one or more reamer features and/or surfaces, as will be discussed in more detail below. A surface of boss feature 320 may be planar with surface 302. Boss feature 320 may extend from connecting edge 310 in a variety of shapes, including but not limited to, quadrilateral (e.g., square, rectangle, diamond), elliptical, circular, and any combination thereof. For example, boss feature 320 may be a combination of quadrilateral and circular. In some embodiments, two boss features 320 may extend from connecting edge 310 and partially traverse engagement feature 306 a predetermined distance. The two boss features 320 may be positioned a predetermined distance from each other.

With specific reference to FIG. 11, boss feature 322 may extend from curved edge 308 and partially traverse engagement feature 306 a predetermined distance. Boss feature 322 may be configured to partially engage with one or more reamer features and/or surfaces, as will be discussed in more detail below. A surface of boss feature 322 may be planar with surface 302. Boss feature 322 may extend from curved edge 308 in a variety of shapes, including but not limited to, quadrilateral (e.g., square, rectangle, diamond), elliptical, circular, and any combination thereof. In some embodiments, two boss features 322 may extend from curved edge 308 and partially traverse engagement feature 306 a predetermined distance. The two boss features 322 may be positioned a predetermined distance from each other. Engagement feature 306 may further define one or more corner engagement features 324. Corner engagement features 324 may interface with one or more surfaces and/or features of a reamer. Engagement feature 306 may define two corner engagement features 324 in close proximity to connecting edge 310.

Caddy system 300 may further define one or more upright features (not shown) positioned with respect to surface 302, as discussed above. Upright feature (not shown) may be configured and dimensioned to interface with sidewall 206 of tray 202. In a non-limiting example, upright feature (not shown) may be used as a handle for moving caddy system 300 and/or to directly/indirectly engage with one or more medical devices. However, additional uses are appreciated. Particularly, one or more surfaces of upright feature may be positioned in close proximity to sidewalls of tray 202. Upright feature (not shown) may further define a feature that extends from upright feature (not shown). Feature (not shown) may extend angularly or perpendicularly from upright feature (not shown). Upright feature (not shown) and feature (not shown), in combination, may define a handle. Feature (not shown) may define additional features for ease of handling and/or description of caddy system 300, including but not limited to, user engagement features (e.g., cut-outs, including square, rectangle, semi-circular, among other shapes) and unique characters (e.g., alphanumeric characters, symbols) and unique colors specific to one or more medical devices. Cut-outs may be positioned on opposing sides for engagement by a user. Upright feature (not shown) and feature (not shown) may extend substantially the width of surface 302. In another embodiment, upright feature (not shown) and feature (not shown) may be larger than or smaller than the width of surface 302. Upright feature (not shown) may, in part, define one or more openings (e.g., slots, engagement features). Slots (not shown) may facilitate pass-through of one or more fluids. For example, fluids associated with central sterile processing.

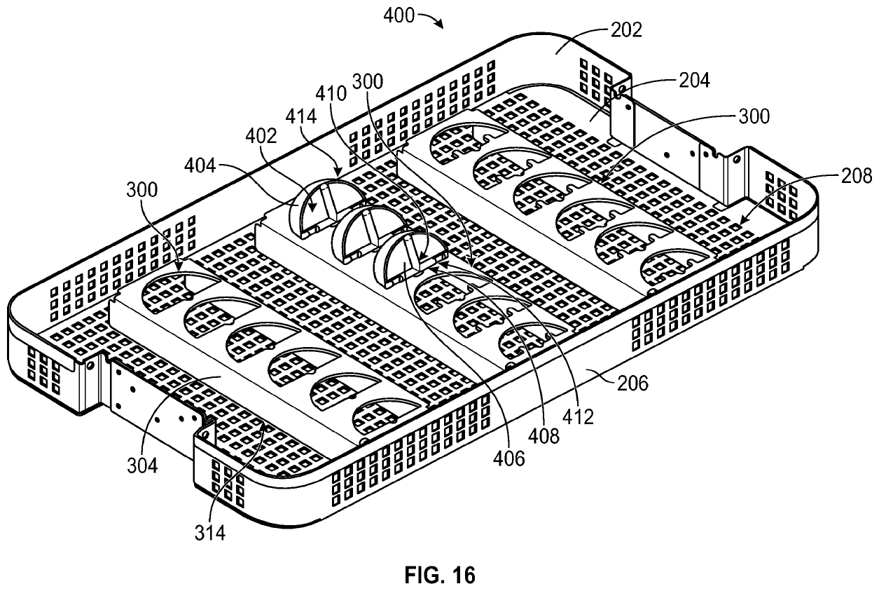

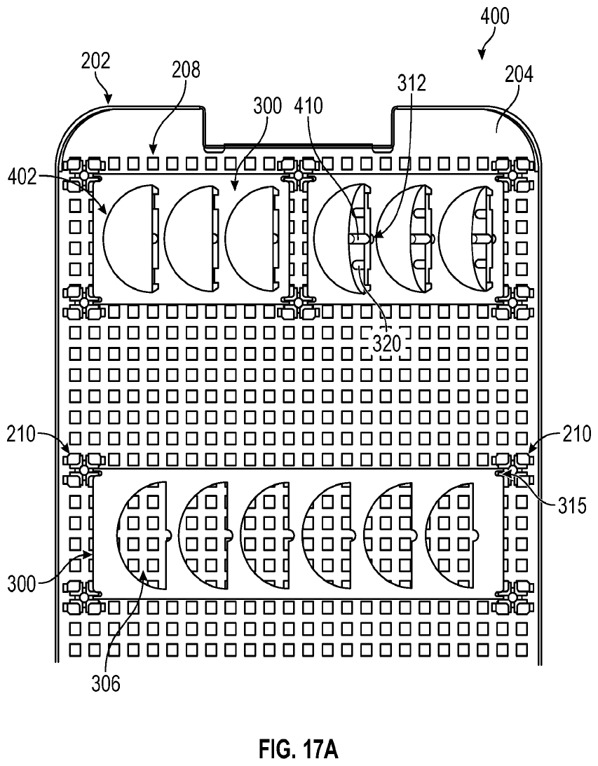

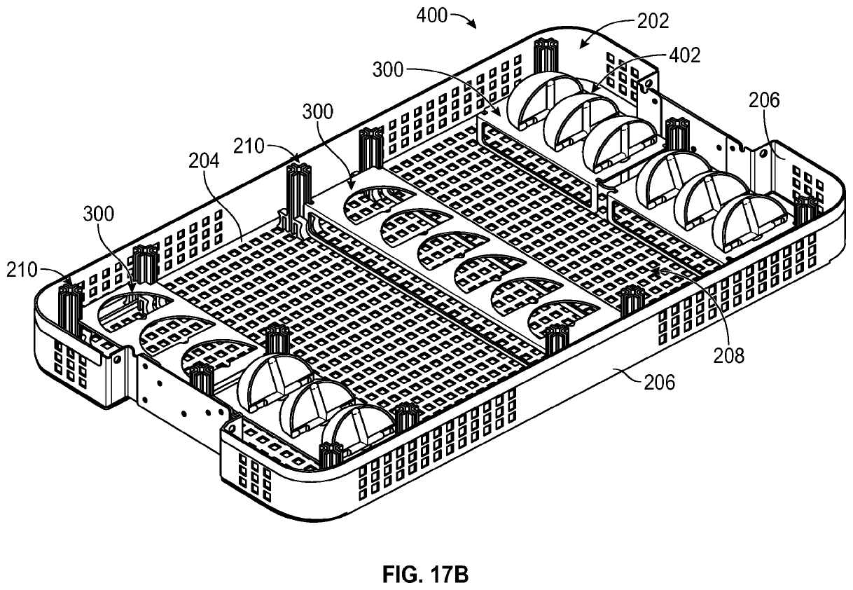

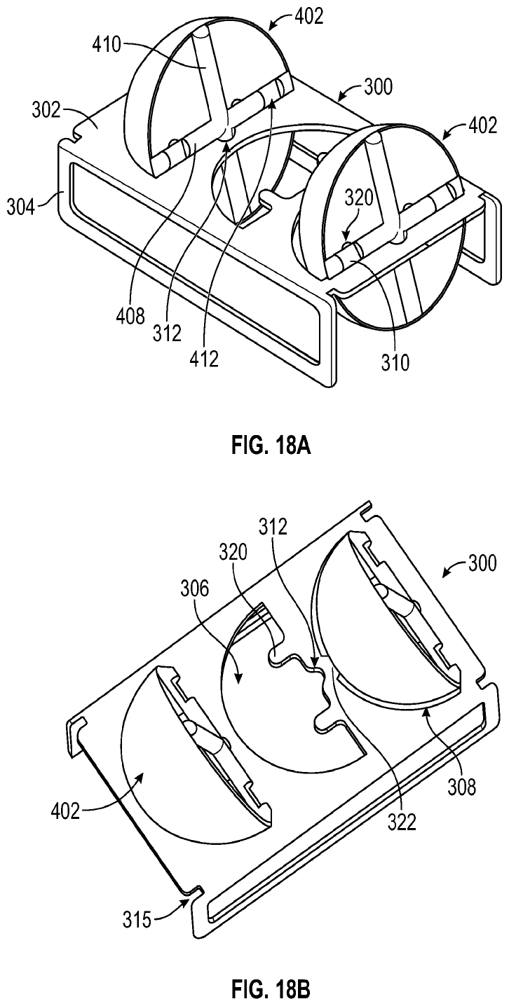

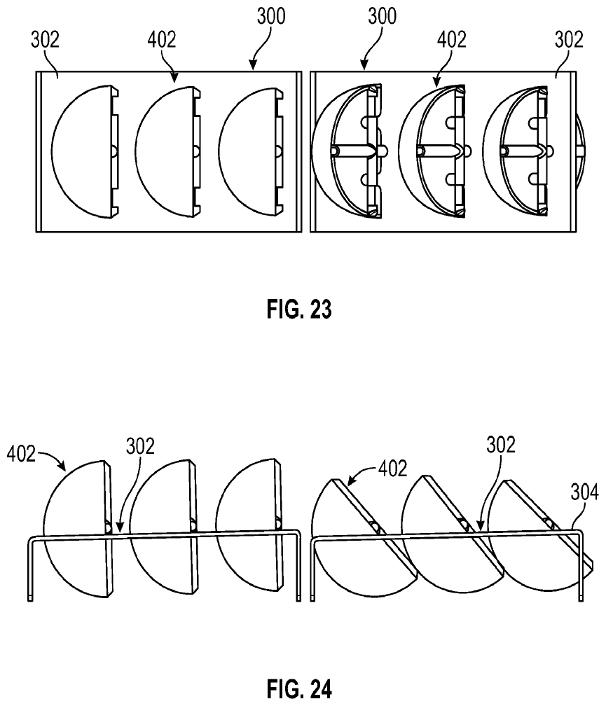

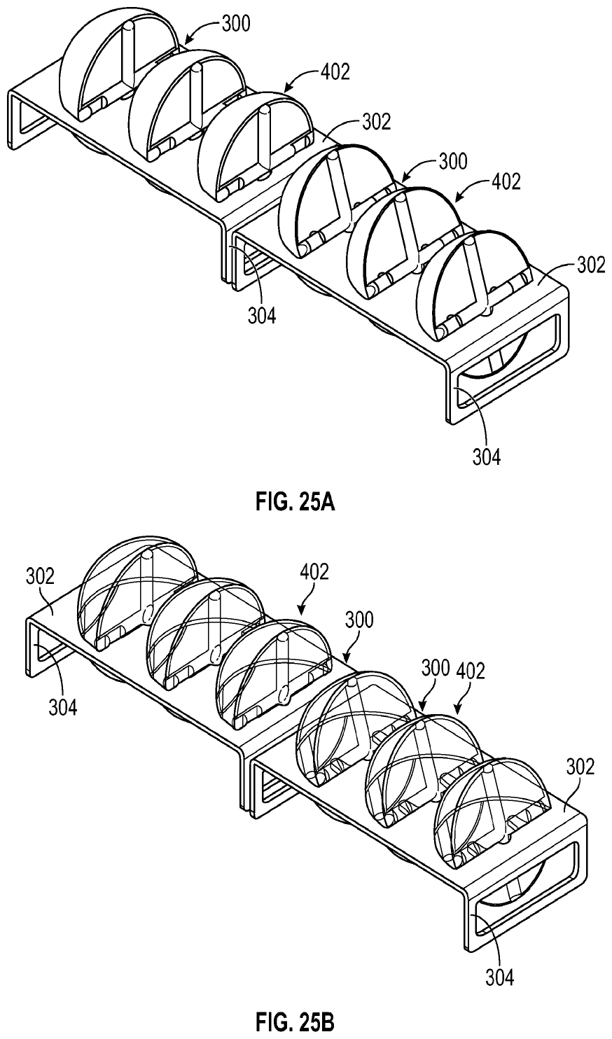

FIGS. 16-25B schematically depict assembly 400 including a plurality of caddy systems 300 positioned adjacent to each other and with respect to tray 202. Although depicted as a plurality of caddy systems 300, one or more cavity systems 300 may be utilized without departing from the spirit/scope of this disclosure. Depending on the situation, tray 202 may be partially filled or completely filled with one or more caddy system(s) 300.

As discussed above, tray 202 includes bottom 204 and sidewalls 206 which partially define an interior. Interior is configured and dimensioned to receive one or more caddy systems 300. Particularly, bottom edge 314 of sidewall 304 of caddy system 300 may be at least partially in direct/indirect contact with bottom 204 of tray 202. More particularly, bottom edge 314 may define features/elements that at least partially interface with bottom 204. For example, features/elements that enable fluid to pass-through (e.g., fluid from the central sterile process) and/or that interface caddy system 300 with bottom 204. Tray 202 further includes a plurality of perforations 208 positioned with respect to bottom 204 and/or sidewalls 206. Perforations 208 may assist with securing one or more caddy system(s) 300 to tray 202. However, caddy system 300 may be positioned within the interior of tray 202 without being affixed either directly/indirectly by one or more perforations 208.

In an exemplary embodiment, caddy system 300 may be positioned within the interior of tray 202. The quantity of caddy systems 300 positioned within tray 202 is may depend on the desired perioperative process and/or central sterile processing process. Thus, one caddy system 300 may be positioned within tray 202 or a plurality of caddy systems 300 may be positioned within tray 202. If two or more caddy systems 300 are positioned within one tray 202, one or more caddy systems 300 may be removed from tray 202 without disturbing one or more of the remaining caddy systems 300. Caddy system 300 may be in direct/indirect contact with one or more sidewalls 206 and bottom 204. Caddy system 300 may be in direct/indirect contact with two sidewalls 206 and bottom 204. Caddy system 300 may be in direct/indirect contact with two sidewalls 206, bottom 204 and at least one adjacent caddy system 300.

Caddy system 300 may be releasably contained within tray 202 by one or more posts 210 positioned with respect to one or more perforations 208. Particularly, one or more posts 210 may be in direct/indirect contact with one or more corners 315 of caddy system 300. In some embodiments, one post 210 may be in direct/indirect contact with at least two caddy systems 300. In other embodiments, one post 210 may be in direct/indirect contact with up to four caddy systems 300. Post 210 and/or caddy system 300 (e.g., corner 315) may include features/elements to facilitate engagement between post 210 and caddy system 300. Reference is made to the above post 210 description.

Caddy system 300 may be configured and dimensioned to interface with one or more medical devices (e.g., reamers, tibial base template, tibial trials/sizers). Caddy system 300 may be configured and dimensioned to interface with a plurality of medical devices (e.g., reamers, tibial base template, tibial trials/sizers). Caddy system 300 may be configured and dimensioned to interface with a plurality of medical devices (e.g., reamers, tibial base template, tibial trials/sizers) of various sizes. The one or more medical devices may be initially positioned within one or more corresponding engagement features and then the one or more medical devices may be pivoted to releasably lock relative to engagement feature 306. The one or more medical devices may be positioned at least partially within engagement feature 306 and may be perpendicular or angled with respect to engagement feature 306 and/or surface 302.

In an exemplary embodiment, caddy system 300 may be configured and dimensioned to interface with one or more reamers 402. Assembly 400 includes caddy system 300, which may be configured and dimensioned to interface with tray 202 and optionally with one or more reamers 402.

Reamer 402 may be a semi-hemispherical cutting tool defining an outer surface 404 and an inner cavity 406. Outer surface 404 may further define a plurality of cutting features (not shown). Reamer 402 may further include one or more bars 408, 410 positioned relative to cavity 406. Bars 408, 410 may be positioned in a substantially cross shape (“plus sign”). However, additional configurations are appreciated. In a non-limiting example, but for ease of explanation, bar 408 may be horizontal and bar 410 may be vertical. Bars 408, 410 may promote stability of reamer 402. Bars 408, 410 may further include one or more surfaces 412 for displaying identifying characteristics. The disclosed identifying characteristics may include, but are not limited to, unique characters (e.g., symbols, alphanumeric characters) and/or corresponding colors. The identifying characteristics may be visible to a user when reamer 402 is positioned at least partially within engagement feature 306.

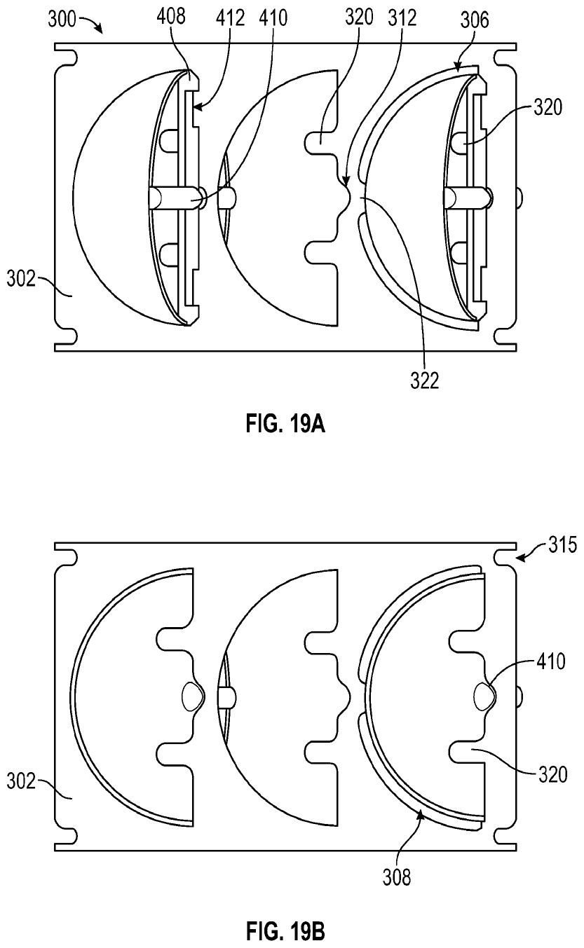

Engagement feature 306 may partially interface with one or more features and/or surfaces of reamer 402, including but not limited to, outer surface 404, inner cavity 406, bars 408, 410, cavity edge 414 and any combination thereof. Reamer 402 may be positioned at least partially within engagement feature 306 and may be perpendicular or angled with respect to engagement feature 306 and/or surface 302. Vertical bar 410 may at least partially engage with recessed portion 312. Horizontal bar 408 may be in at least partial contact with one or more boss features 320. Horizontal bar 408 may be in at least partial contact with surface 302 of one or more boss features 320. Outer surface 404 may be in at least partial contact with curved edge 308 and/or one or more boss features 322. Outer surface 404 having one or more cutting features may be in at least partial contact with one or more boss features so as to avoid damaging the one or more cutting features, as depicted in FIGS. 19A and 19B. Cavity edge 414 may be in at least partial contact with one or more corner engagement features 324.

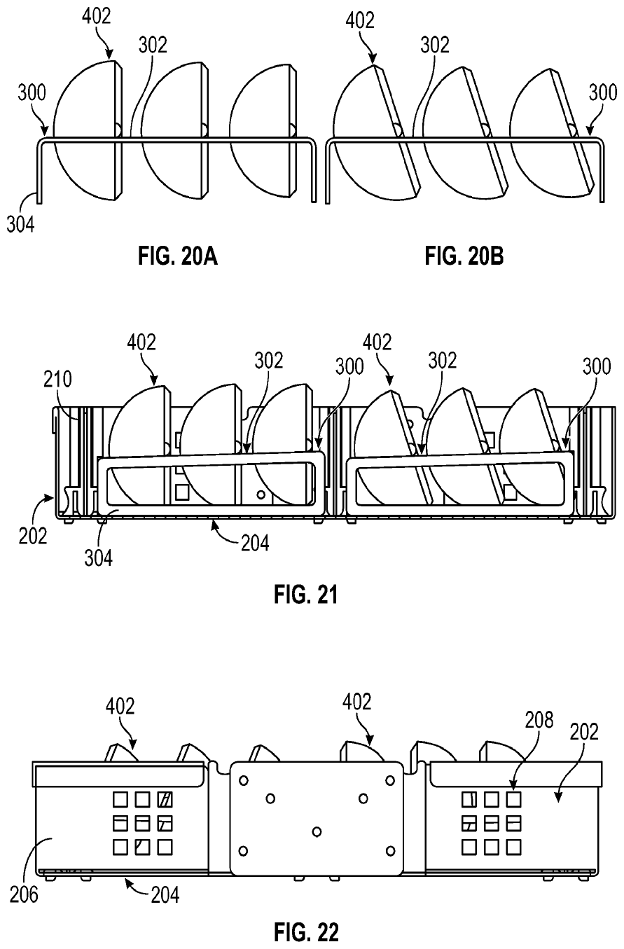

Reamer 402 may be positioned within corresponding engagement feature 306. Reamer 402 may be positioned within corresponding engagement feature 306 in a substantially perpendicular configuration. In one embodiment, reamer 402 may be initially positioned within corresponding engagement feature 306. Reamer 402 may be initially positioned within corresponding engagement feature 306 in a substantially perpendicular configuration, as depicted in FIG. 20A. Then, reamer 402 may be pivoted relative to engagement feature 306 and/or surface 302 to releasably lock reamer 402 relative to engagement feature 306 and/or surface 302, as depicted in FIG. 20B. Reamer 402 may be releasably locked relative to engagement feature 306, in part, by recessed portion 312 being in at least partial contact with vertical bar 410, boss feature 320 being in at least partial contact with horizontal bar 408 and/or outer surface 404 being in at least partial contact with curved edge 308 or boss feature 322. In one example, the one or more reamers may be locked with respect to the disclosed caddy system to ensure the one or more reamers remain positioned with respect to caddy system 300 during transportation.

Caddy system 300 may be configured and dimensioned so as to suspend medical devices a distance above bottom 204. Positioning reamers 402 on an angle enables larger reamers 402 to fit substantially with the interior of tray 202 such that a lid may be positioned on top of tray 202. Positioning reamers 402 perpendicularly or angularly, as described above, promotes better cleaning and sterilization, a shortcoming of current packaging configurations (e.g., medical devices positioned laying down).

Caddy system 10, 50, 300 may be removed from tray 202 for use as a separate “tool box”. The separate tool box may include medical devices that compliment each other and/or that are apart of a set. Caddy system 10, 50, 300 may be customized to a user's preference (e.g., to include a plurality of medical device sizes). In an exemplary embodiment, a plurality of caddy systems 10, 50, 300 may be positioned within one tray. The plurality of caddy systems may be configured and adapted to retain a plurality of variously-sized medical devices (e.g., femoral trial, reamer, tibial base template, tibial trials/sizers). If two or more caddy systems 10, 50, 300 are positioned within one tray 202, one or more caddy systems 10, 50, 300 may be removed from tray 202 without disturbing one or more of the remaining caddy systems 10, 50, 300.

In one non-limiting example, a plurality of caddy systems 10, 50, 300 are positioned within tray 202 for use during surgery. During surgery, one or more caddy systems 10, 50, 300 may be removed from tray 202 and delivered to the operating location, while tray 202 remains stationary. Delivery of the one or more caddy systems 10, 50, 300 provides ease of transportation by a user with a reduced risk in dropping one or more medical devices.

In part, caddy systems 10, 50, 300 promote efficiency during a patient's surgical procedure (e.g., during the perioperative process, which may include preoperative care, intraoperative care, and postoperative care) and/or during the central sterile processing process (e.g., autoclave-based steam sterilization).

The disclosed caddy system 10, 50, 300 may be fabricated from one or more materials that is/are impervious to the harsh conditions of central sterile processing processes. However, materials that are not impervious to the conditions of central sterile processing processes may be coated so as to make them resistant, i.e., able to withstand sterilization conditions (e.g., coating, anodizing, and/or plating). The disclosed caddy system 10, 50, 300 may be fabricated from plastic (e.g., thermoplastic), silicone, or metal (e.g., aluminum).

Although the present disclosure has been described with reference to exemplary implementations, the present disclosure is not limited by or to such exemplary implementations. Rather, various modifications, refinements and/or alternative implementations may be adopted without departing from the spirit or scope of the present disclosure.

1. A caddy system, comprising:
a body having a first surface and a second surface; and
at least one engagement feature incorporated with at least one of the first surface and the second surface, wherein the at least one engagement feature is configured and adapted to releasably interface with at least one medical device.

2. The caddy system of claim 1 further comprising at least one post attachment feature configured and adapted to interface with at least one post.

3. The caddy system of claim 1, wherein the at least one engagement feature further defines an opening, wherein the opening extends through the first surface and the second surface of the body.

4. The caddy system of claim 3, wherein the opening is configured and adapted to at least partially retain at least one medical device.

5. The caddy system of claim 3, wherein the at least one engagement feature further defines one or more boss features that at least partially traverse the opening.

6. The caddy system of claim 5, wherein the at least one engagement feature defines a first boss feature and two oppositely positioned second boss features, wherein the boss features are configured and adapted to interface with one or more surfaces of the at least one medical device.

7. The caddy system of claim 1, wherein a plurality of engagement features are incorporated with at least one of the first surface and the second surface.

8. The caddy system of claim 1, wherein the body further defines at least one handle.

9. The caddy system of claim 8, wherein the at least one handle is configured and adapted to at least partially interface with a tray.

10. The caddy system of claim 8, wherein the body and the at least one handle are sized to substantially extend between two sidewalls of a tray.

11. The caddy system of claim 1, wherein the body further defines at least one sidewall configured and adapted to at least partially interface with a tray.

12. The caddy system of claim 11, wherein a first portion of the at least one sidewall is configured at a first position relative to the body and a second portion of the at least one sidewall is configured at a second position relative to the body.

13. The caddy system of claim 12, wherein the first portion is substantially perpendicular with respect to the body and the second portion is substantially angled with respect to the body.

14. The caddy system of claim 13, wherein the second portion further defines a plurality of cavities configured to at least partially engage with one or more medical devices.

15. A method of retaining a medical device with a caddy system, comprising:
positioning at least one medical device to a first position within a corresponding engagement feature; and
repositioning the at least one medical device to a second position within the corresponding engagement feature,
wherein at the second position, the at least one medical device is releasably locked to the corresponding engagement feature.

16. The method of claim 15, wherein the at least one medical device is pivoted into the second position.

17. The method of claim 15, wherein the at least one medical device is positioned substantially perpendicular with respect to the corresponding engagement feature when configured in the first position and the at least one medical device is pivoted to be angled with respect to the corresponding engagement feature when configured in the second position.

18. A caddy system, comprising:
a body having a first surface and a second surface;
at least two sidewalls extending from the second surface of the body, wherein the body and the at least two sidewalls define a substantially U-shaped configuration; and
at least one engagement feature incorporated with at least one of the first surface and the second surface, wherein the at least one engagement feature further defines at least one opening extending through the first and second surfaces, and
wherein the at least one engagement feature is configured and adapted to releasably interface with at least one medical device.

19. The caddy system of claim 18 further comprising at least one handle extending from the first surface of the body opposite the at least two sidewalls.

20. The caddy system of claim 18, wherein the at least one engagement feature further defines one or more boss features that at least partially traverse the opening.
